SEQUENCE LISTING (8 sequences)

SEQ ID NO: 1 - DNA, 1389 bp, Homo sapiens
atgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagctga

SEQ ID NO: 2 - protein, 462 aa, Homo sapiens
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSS

SEQ ID NO: 3 - DNA, 1236 bp, Homo sapiens
atgtctcccgcccggctccggccccgactgcacttctgcctggtcctgttgctgctgctggtggtgccggcggcatggggctgcgggccgggtcgggtggtgggcagccgccggcgaccgccacgcaaactcgtgccgctcgcctacaagcagttcagccccaatgtgcccgagaagaccctgggcgccagcggacgctatgaaggcaagatcgctcgcagctccgagcgcttcaaggagctcacccccaattacaatccagacatcatcttcaaggacgaggagaacacaggcgccgaccgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagcctcacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggactccgggggagggtgtgcattggtacccccagctgctctaccgcctggggcgtctcctgctagaagagggcagcttccacccactgggcatgtccggggcagggagctga

SEQ ID NO: 4 - protein, 411 aa, Homo sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 5 - DNA, 1191 bp, Homo sapiens
atggctctcctgaccaatctactgcccctgtgctgcttggcacttctggcgctgccagcccagagctgcgggccgggccgggggccggttggccggcgccgctatgcgcgcaagcagctcgtgccgctactctacaagcaatttgtgcccggcgtgccagagcggaccctgggcgccagtgggccagcggaggggagggtggcaaggggctccgagcgcttccgggacctcgtgcccaactacaaccccgacatcatcttcaaggatgaggagaacagtggagccgaccgcctgatgaccgagcgttgtaaggagcgggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggctgctttccgggaaatgcaactgtgcgcctgtggagcggcgagcggaaagggctgcgggaactgcaccgcggagactgggttttggcggccgatgcgtcaggccgggtggtgcccacgccggtgctgctcttcctggaccgggacttgcagcgccgggcttcatttgtggctgtggagaccgagtggcctccacgcaaactgttgctcacgccctggcacctggtgtttgccgctcgagggccggcgcccgcgccaggcgactttgcaccggtgttcgcgcgccggctacgcgctggggactcggtgctggcgcccggcggggatgcgcttcggccagcgcgcgtggcccgtgtggcgcgggaggaagccgtgggcgtgttcgcgccgctcaccgcgcacgggacgctgctggtgaacgatgtcctggcctcttgctacgcggttctggagagtcaccagtgggcgcaccgcgcttttgcccccttgagactgctgcacgcgctaggggcgctgctccccggcggggccgtccagccgactggcatgcattggtactctcggctcctctaccgcttagcggaggagctactgggctga

SEQ ID NO: 6 - protein, 396 aa, Homo sapiens
MALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

SEQ ID NO: 7 - DNA, 22 bp, Homo sapiens
tgctctcccagttctcagactc

SEQ ID NO: 8 - DNA, 18 bp, Homo sapiens
ccacaaccttggctttgg

(19) United States
(12) Patent Application Publication
(10) Pub. No.: US 2015/0037350 A1
(43) Pub. Date: Feb. 5, 2015

(54) NEGATIVE MODULATOR OF HEDGEHOG SIGNALLING FOR USE IN TREATING TH2-MEDIATED DISEASES

(71) Applicant: UCL.Business PLC, London (GB)

(72) Inventors: Anna L. Furmanski, London (GB); Jose Ignacio Saldana, London (GB); Tessa Crompton, London (GB)

(73) Assignee: UCL BUSINESS PLC, London (GB)

(21) Appl. No.: 14/385,251

(22) PCT Filed: Mar. 15, 2013
(86) PCT No.: PCT/GB2013/050660

§ 371 Date: Sep. 15, 2014

(30) Foreign Application Priority Data
Mar. 16, 2012 (GB) 1204645.4

(51) Int. Cl. C07K 16/22; A61K 39/00; A61K 38/18

(52) U.S. Cl. CPC C07K 16/22; A61K 38/18; A61K 39/00
USPC 424/158.1; 514/21.2; 514/13.2; 514/16.6; 514/17.9; 514/20.8; 424/184.1; 514/16.8; 514/1.8

(57) ABSTRACT

The invention provides means for treating Th1- and Th2-mediated diseases, such as asthma, allergic dermatitis and Th2-driven cancer. The invention extends to pharmaceutical compositions for use in treating such conditions, and to methods of treatment. The invention also extends to adjuvants and vaccines per se, and to their use in enhancing the immunomodulatory activity of immunogens.

The present invention relates to the treatment of diseases, including Th1- and Th2-mediated diseases, and particularly, although not exclusively, to the treatment of asthma, allergic dermatitis and Th2-driven cancer. The invention also extends to pharmaceutical compositions for use in treating such conditions, and to methods of treatment. The invention also extends to adjuvants and vaccines per se, and to their use in enhancing the immunomodulatory activity of immunogens.

Asthma is a chronic, Th-2 mediated inflammatory disease of the airways, thought to be caused by a combination of genetic and environmental factors. Symptoms can be treated with an inhaled β2 agonist, such as salbutamol, or by corticosteriods. However, unfortunately, the use of steroids comes with a number of significant problems. Firstly, they do not always work, and acute asthma exacerbations can still be fatal, despite steroid therapy. Secondly, some patients are ‘steroid resistant’, and so they do not respond to steroid therapy. Thirdly, some patients become steroid-dependent, and so they cannot be taken off steroids and even require increases in dose. Fourthly, steroids function as long-term non-specific immunosuppressants, and have significant side-effects (e.g. low bone-density, cataract, obesity and weakened immunity), when used long-term either systemically or topically. In view of these problems with using steroids, it is important to have alternative and additional treatments, particularly for treating Th-2 mediated diseases such as asthma, which represent a huge health and economic burden. To date, attempts to target cytokines (eg. anti-IL4) have been unsuccessful, and have given conflicting results in clinical trials, and so new approaches and targets are needed.

Hedgehog (Hh) family proteins are secreted inter-cellular signalling molecules, essential for organogenesis during embryonic development, and homeostasis of adult tissues. Morphogens, such as Hh, specify cell fate and patterning by establishing a concentration gradient, where the position of a target cell relative to the source of Hh determines the signal received. Inappropriate activation of the Hh signalling pathway leads to several cancers and Hh has recently been shown to be involved in the pathogenesis of haematological and lymphoid malignancies. Knockout and transgenic mouse models show that Hh signalling is important in regulating thymocyte development, promoting differentiation of the earliest T-cell progenitors, but negatively regulating later differentiation and selection.

The Hh family proteins, Sonic Hh (Shh), Indian Hh (Ihh) and Desert Hh (Dhh) share a common signalling pathway, which is initiated by binding to the cell-surface receptor Patched 1 (Ptch1), relieving inhibition of Smoothened (Smo), resulting in signal transduction. At the end of the pathway are the Gli family of transcription factors (Gli1, Gli2 and Gli3). Gli proteins bind DNA at consensus Gli-family binding sites and directly modulate target gene transcription. Gli2 is necessary to initiate the signal and acts mainly as an activator, but can be processed to activate or repress transcription by post-translational modification. Strength and duration of the signal received is determined by the cellular balance of intracellular Gli-Repressor and Gli-Activator protein forms.

Peripheral T-cells express components of the Hh signalling pathway, and Shh is involved in the regulation of T-cell activation in vitro. Hh family proteins are widely expressed in the stroma/epithelia of postnatal tissues, many of which harbour resident T-cells, including skin, lung, gut, bone marrow and spleen. Naïve CD4+ T-cells can differentiate down various lineage pathways, with distinct T-helper (Th) functions. These lineage fate decisions are controlled by specific transcriptional programs, which are thought to be initiated during antigen priming and depend on the establishment of feedback loops to enhance lineage-specific cytokine production. Th1 and Th2 cells can be distinguished by their hallmark profiles of cytokine secretion, expression of lineage-specific transcription factors and different cellular functions.

Th1 cells express Tbet, produce interferon gamma (IFNγ) and control immune responses against intracellular pathogens. The cells, on the other hand, express Gata3, secrete interleukin-4 (IL-4), IL-5, IL-9, IL-13 and IL-25 and are important for protection against extracellular parasites. The cells are also involved in the pathogenesis of allergic inflammation and atopic disease. Both IL-4 and Gata3 are required for the differentiation of Th2 cells. IL-4 is the primary Th2 cytokine and is necessary for the generation of IL-5, IL-9 and IL-10 and Th2-driven immune responses (Kopf, M. et al. Nature 362, 245-248, 1993). Conditional deletion of Gata3 from naive T-cells blocked differentiation into functional IL-4-secreting cells. Expression of Gata3 and Il4 are closely linked: Gata3 can directly activate transcription of the Il4 gene, but for Th2 differentiation, naive T-cells require TCR and IL-4 signalling for strong induction of Gata3. Once established, this creates a positive feedback loop, but the factors that induce initial upregulation of both of these key genes are incompletely understood. TCR signal strength may influence Th lineage decisions, but regulation of induction of differentiation is complex, and both environmental and cell-intrinsic mechanisms determine transcriptional profile, lineage fate, plasticity and function. Although much research has investigated the role of cytokines in determining T-cell differentiation, very little is known about the function of other non-immune factors, such as Hh, which are secreted from cells into their environment.

The inventors set out to test their hypothesis that Hh influences mature CD4+ helper-cell (Th) differentiation. As described in the Examples, the inventors are the first to demonstrate that the Th2-specifying cytokine, Il4, is a novel downstream target of Hh signalling in mature T-cells. They surprisingly found that Hh-dependent transcription promotes Th2 differentiation in vitro and pathology in vivo, and that expression of Hh ligand in tissue increased following induction of allergic disease. The inventors have shown therefore that resident T-cell responses can be skewed by Hh secreted from inflamed or remodelling tissue, or tumours. These data thus present a novel mechanism for the dynamic regulation of immune responses by local microenvironments, with implications for allergy and cancer immunity, and can be used to treat either Th1 or Th2-mediated diseases.

Thus, according to a first aspect of the invention, there is provided a modulator of Hedgehog (Hh) signalling, for use in the treatment, amelioration or prevention of a Th1- or Th2-mediated disease.

In a second aspect, there is provided a method of treating, ameliorating or preventing a Th1- or Th2-mediated disease in a subject, the method comprising administering, to a subject in need of such treatment, a therapeutically effective amount of a modulator of Hedgehog (Hh) signalling.

Naïve CD4 T-cells can be instructed to differentiate down various lineage pathways, each of which exhibit distinct T-helper (Th) functions. These lineage fate decisions are controlled by distinct transcriptional programs, dependent both on establishment of lineage-specific cytokine production and largely uncharacterised microenvironmental influences. The inventors have surprisingly demonstrated that Hedgehog (Hh) signalling skewed CD4 cell differentiation toward the Th2 lineage via upregulation of the previously unknown Hh-target gene, Il4, and subsequent increased expression of Gata3, whereas repressing or reducing Hh signalling impaired Gata3 induction and Th2 differentiation. Furthermore, they have also shown that Hh-dependent transcription in T-cells increased the severity of Th2-associated pathology in the murine model of asthma, and is thus important in regulating T-cell differentiation and function in vivo. The also showed that Hh family proteins are detectable in wild-type adult lung tissue, and that expression of Shh transcript and protein increased following induction of allergic inflammation. Thus, the inventors postulate that local adaptive immune responses can be skewed by Hh released from damaged/inflamed tissue, providing a novel mechanism for dynamic tissue-dependent immune regulation by the stroma/epithelium.

There are currently three known mammalian Hedgehog (Hh) family proteins, i.e. Sonic Hh (Shh), Indian Hh (Ihh) and Dhh. Accordingly, the modulator may be capable of modulating Sonic Hh (Shh), Indian Hh (Ihh) and/or Desert Hh (Dhh) signalling. Preferably, however, the modulator is capable of modulating all Hh signalling.

The cDNA sequence (1389 nucleotides) of human Sonic Hh (Shh), having Transcript ID: CCDS5942.1, is provided herein as SEQ ID No:1, as follows.

[SEQ ID No: 1]

ATGCTGCTGCTGGCGAGATGTCTGCTGCTAGTCCTCGTCTCCTCGCTGC
TGGTATGCTCGGGACTGGCGTGCGGACCGGGCAGGGGGTTCGGGAAGAG
GAGGCACCCCAAAAAGCTGACCCCTTTAGCCTACAAGCAGTTTATCCCC
AATGTGGCCGAGAAGACCCTAGGCGCCAGCGGAAGGTATGAAGGGAAGA
TCTCCAGAAACTCCGAGCGATTTAAGGAACTCACCCCCAATTACAACCC
CGACATCATATTTAAGGATGAAGAAAACACCGGAGCGGACAGGCTGATG
ACTCAGAGGTGTAAGGACAAGTTGAACGCTTTGGCCATCTCGGTGATGA
ACCAGTGGCCAGGAGTGAAACTGCGGGTGACCGAGGGCTGGGACGAAGA
TGGCCACCACTCAGAGGAGTCTCTGCACTACGAGGGCCGCGCAGTGGAC
ATCACCACGTCTGACCGCGACCGCAGCAAGTACGGCATGCTGGCCCGCC
TGGCGGTGGAGGCCGGCTTCGACTGGGTGTACTACGAGTCCAAGGCACA
TATCCACTGCTCGGTGAAAGCAGAGAACTCGGTGGCGGCCAAATCGGGA
GGCTGCTTCCCGGGCTCGGCCACGGTGCACCTGGAGCAGGGCGGCACCA
AGCTGGTGAAGGACCTGAGCCCCGGGGACCGCGTGCTGGCGGCGGACGA
CCAGGGCCGGCTGCTCTACAGCGACTTCCTCACTTTCCTGGACCGCGAC
GACGGCGCCAAGAAGGTCTTCTACGTGATCGAGACGCGGGAGCCGCGCG
AGCGCCTGCTGCTCACCGCCGCGCACCTGCTCTTTGTGGCGCCGCACAA
CGACTCGGCCACCGGGGAGCCCGAGGCGTCCTCGGGCTCGGGGCCGCCT
TCCGGGGGCGCACTGGGGCCTCGGGCGCTGTTCGCCAGCCGCGTGCGCC
CGGGCCAGCGCGTGTACGTGGTGGCCGAGCGTGACGGGGACCGCCGGCT
CCTGCCCGCCGCTGTGCACAGCGTGACCCTAAGCGAGGAGGCCGCGGGC
GCCTACGCGCCGCTCACGGCCCAGGGCACCATTCTCATCAACCGGGTGC
TGGCCTCGTGCTACGCGGTCATCGAGGAGCACAGCTGGGCGCACCGGGC
CTTCGCGCCCTTCCGCCTGGCGCACGCGCTCCTGGCTGCACTGGCGCCC
GCGCGCACGGACCGCGGCGGGGACAGCGGCGGCGGGGACCGCGGGGGCG
GCGGCGGCAGAGTAGCCCTAACCGCTCCAGGTGCTGCCGACGCTCCGGG
TGCGGGGGCCACCGCGGGCATCCACTGGTACTCGCAGCTGCTCTACCAA
ATAGGCACCTGGCTCCTGGACAGCGAGGCCCTGCACCCGCTGGGCATGG
CGGTCAAGTCCAGCTGA

The protein sequence (462 amino acids) of human Sonic Hh (Shh), having ENST00000297261, is provided herein as SEQ ID No:2, as follows.

[SEQ ID No: 2]

MLLLARCLLLVLVSSLLVCSGLACGPGRGFGKRRHPKKLTPLAYKQFIP
NVAEKTLGASGRYEGKISRNSERFKELTPNYNPDIIFKDEENTGADRLM
TQRCKDKLNALAISVMNQWPGVKLRVTEGWDEDGHHSEESLHYEGRAVD
ITTSDRDRSKYGMLARLAVEAGFDWVYYESKAHIHCSVKAENSVAAKSG
GCFPGSATVHLEQGGTKLVKDLSPGDRVLAADDQGRLLYSDFLTFLDRD
DGAKKVFYVIETREPRERLLLTAAHLLFVAPHNDSATGEPEASSGSGPP
SGGALGPRALFASRVRPGQRVYVVAERDGDRRLLPAAVHSVTLSEEAAG
AYAPLTAQGTILINRVLASCYAVIEEHSWAHRAFAPFRLAHALLAALAP
ARTDRGGDSGGGDRGGGGGRVALTAPGAADAPGAGATAGIHWYSQLLYQ
IGTWLLDSEALHPLGMAVKSS

The cDNA sequence (1236 nucleotides) of human Indian Hh(Ihh), having Transcript ID: CCDS33380.1, is provided herein as SEQ ID No:3, as follows.

[SEQ ID No: 3]

ATGTCTCCCGCCCGGCTCCGGCCCCGACTGCACTTCTGCCTGGTCCTGT
TGCTGCTGCTGGTGGTGCCGGCGGCATGGGGCTGCGGGCCGGGTCGGGT
GGTGGGCAGCCGCCGGCGACCGCCACGCAAACTCGTGCCGCTCGCCTAC
AAGCAGTTCAGCCCCAATGTGCCCGAGAAGACCCTGGGCGCCAGCGGAC
GCTATGAAGGCAAGATCGCTCGCAGCTCCGAGCGCTTCAAGGAGCTCAC
CCCCAATTACAATCCAGACATCATCTTCAAGGACGAGGAGAACACAGGC
GCCGACCGCCTCATGACCCAGCGCTGCAAGGACCGCCTGAACTCGCTGG
CTATCTCGGTGATGAACCAGTGGCCCGGTGTGAAGCTGCGGGTGACCGA
GGGCTGGGACGAGGACGGCCACCACTCAGAGGAGTCCCTGCATTATGAG
GGCCGCGCGGTGGACATCACCACATCAGACCGCGACCGCAATAAGTATG
GACTGCTGGCGCGCTTGGCAGTGGAGGCCGGCTTTGACTGGGTGTATTA
CGAGTCAAAGGCCCACGTGCATTGCTCCGTCAAGTCCGAGCACTCGGCC
GCAGCCAAGACGGGCGGCTGCTTCCCTGCCGGAGCCCAGGTACGCCTGG
AGAGTGGGGCGCGTGTGGCCTTGTCAGCCGTGAGGCCGGGAGACCGTGT
GCTGGCCATGGGGGAGGATGGGAGCCCCACCTTCAGCGATGTGCTCATT
TTCCTGGACCGCGAGCCTCACAGGCTGAGAGCCTTCCAGGTCATCGAGA
CTCAGGACCCCCCACGCCGCCTGGCACTCACACCCGCTCACCTGCTCTT
TACGGCTGACAATCACACGGAGCCGGCAGCCCGCTTCCGGGCCACATTT
GCCAGCCACGTGCAGCCTGGCCAGTACGTGCTGGTGGCTGGGGTGCCAG
GCCTGCAGCCTGCCCGCGTGGCAGCTGTCTCTACACACGTGGCCCTCGG
GGCCTACGCCCCGCTCACAAAGCATGGGACACTGGTGGTGGAGGATGTG
GTGGCATCCTGCTTCGCGGCCGTGGCTGACCACCACCTGGCTCAGTTGG
CCTTCTGGCCCCTGAGACTCTTTCACAGCTTGGCATGGGGCAGCTGGAC
TCCGGGGGAGGGTGTGCATTGGTACCCCCAGCTGCTCTACCGCCTGGGG
CGTCTCCTGCTAGAAGAGGGCAGCTTCCACCCACTGGGCATGTCCGGGG
CAGGGAGCTGA

The protein sequence (411 amino acids) of human Indian Hh (Ihh), having ENST00000295731, is provided herein as SEQ ID No:4, as follows.

[SEQ ID No: 4]

MSPARLRPRLHFCLVLLLLLVVPAAWGCGPGRVVGSRRRPPRKLVPLAY
KQFSPNVPEKTLGASGRYEGKIARSSERFKELTPNYNPDIIFKDEENTG
ADRLMTQRCKDRLNSLAISVMNQWPGVKLRVTEGWDEDGHHSEESLHYE
GRAVDITTSDRDRNKYGLLARLAVEAGFDWVYYESKAHVHCSVKSEHSA
AAKTGGCFPAGAQVRLESGARVALSAVRPGDRVLAMGEDGSPTFSDVLI
FLDREPHRLRAFQVIETQDPPRRLALTPAHLLFTADNHTEPAARFRATF
ASHVQPGQYVLVAGVPGLQPARVAAVSTHVALGAYAPLTKHGTLVVEDV
VASCFAAVADHHLAQLAFWPLRLFHSLAWGSWTPGEGVHWYPQLLYRLG
RLLLEEGSFHPLGMSGAGS

The cDNA sequence (1191 nucleotides) of human Desert Hh (Dhh), having Transcript ID: CCDS8779.1, is provided herein as SEQ ID No:5, as follows.

[SEQ ID No: 5]

ATGGCTCTCCTGACCAATCTACTGCCCCTGTGCTGCTTGGCACTTCTGG
CGCTGCCAGCCCAGAGCTGCGGGCCGGGCCGGGGGCCGGTTGGCCGGCG
CCGCTATGCGCGCAAGCAGCTCGTGCCGCTACTCTACAAGCAATTTGTG
CCCGGCGTGCCAGAGCGGACCCTGGGCGCCAGTGGGCCAGCGGAGGGGA
GGGTGGCAAGGGGCTCCGAGCGCTTCCGGGACCTCGTGCCCAACTACAA
CCCCGACATCATCTTCAAGGATGAGGAGAACAGTGGAGCCGACCGCCTG
ATGACCGAGCGTTGTAAGGAGCGGGTGAACGCTTTGGCCATTGCCGTGA
TGAACATGTGGCCCGGAGTGCGCCTACGAGTGACTGAGGGCTGGGACGA
GGACGGCCACCACGCTCAGGATTCACTCCACTACGAAGGCCGTGCTTTG
GACATCACTACGTCTGACCGCGACCGCAACAAGTATGGGTTGCTGGCGC
GCCTCGCAGTGGAAGCCGGCTTCGACTGGGTCTACTACGAGTCCCGCAA
CCACGTCCACGTGTCGGTCAAAGCTGATAACTCACTGGCGGTCCGGGCG
GGCGGCTGCTTTCCGGGAAATGCAACTGTGCGCCTGTGGAGCGGCGAGC
GGAAAGGGCTGCGGGAACTGCACCGCGGAGACTGGGTTTTGGCGGCCGA
TGCGTCAGGCCGGGTGGTGCCCACGCCGGTGCTGCTCTTCCTGGACCGG
GACTTGCAGCGCCGGGCTTCATTTGTGGCTGTGGAGACCGAGTGGCCTC
CACGCAAACTGTTGCTCACGCCCTGGCACCTGGTGTTTGCCGCTCGAGG
GCCGGCGCCCGCGCCAGGCGACTTTGCACCGGTGTTCGCGCGCCGGCTA
CGCGCTGGGGACTCGGTGCTGGCGCCCGGCGGGGATGCGCTTCGGCCAG
CGCGCGTGGCCCGTGTGGCGCGGGAGGAAGCCGTGGGCGTGTTCGCGCC
GCTCACCGCGCACGGGACGCTGCTGGTGAACGATGTCCTGGCCTCTTGC
TACGCGGTTCTGGAGAGTCACCAGTGGGCGCACCGCGCTTTTGCCCCCT
TGAGACTGCTGCACGCGCTAGGGGCGCTGCTCCCCGGCGGGGCCGTCCA
GCCGACTGGCATGCATTGGTACTCTCGGCTCCTCTACCGCTTAGCGGAG
GAGCTACTGGGCTGA

The protein sequence (396 amino acids) of human Desert Hh (Dhh), having ENST00000266991, is provided herein as SEQ ID No:6, as follows.

[SEQ ID No: 6]

MALLTNLLPLCCLALLALPAQSCGPGRGPVGRRRYARKQLVPLLYKQFV
PGVPERTLGASGPAEGRVARGSERFRDLVPNYNPDIIFKDEENSGADRL
MTERCKERVNALAIAVMNMWPGVRLRVTEGWDEDGHHAQDSLHYEGRAL
DITTSDRDRNKYGLLARLAVEAGFDWVYYESRNHVHVSVKADNSLAVRA
GGCFPGNATVRLWSGERKGLRELHRGDWVLAADASGRVVPTPVLLFLDR
DLQRRASFVAVETEWPPRKLLLTPWHLVFAARGPAPAPGDFAPVFARRL
RAGDSVLAPGGDALRPARVARVAREEAVGVFAPLTAHGTLLVNDVLASC
YAVLESHQWAHRAFAPLRLLHALGALLPGGAVQPTGMHWYSRLLYRLAE
ELLG

Underlined nucleotides and amino acids denote alternate exons, and bold residue indicates amino acid encoded across a splice junction.

As shown in the Examples, the inventors have demonstrated that Hh signalling induced IL4, and thereby promotes differentiation to the Th2 lineage. Based on this knowledge, the inventors have realised that it is possible to use, in one embodiment, a negative modulator of Hh signalling in order to promote a switch away from a Th2 response, and thereby treat Th2-mediated disease. Conversely, in another embodiment, it is possible to use a positive modulator of Hh signalling to promote a switch away from a Th1 response, and thereby treat Th1-mediated disease.

Thus, in one embodiment, the modulator may be a negative modulator of Hedgehog (Hh) signalling (for example an antagonist), for use in treating a Th2-mediated disease. The negative modulator may be capable of:—
(i) altering the conformational state of the receptors or signal transduction molecules through which Hh signalling is achieved, for example by destabilizing the active conformation of that receptor and/or maintaining the receptor in its inactive conformation to thereby prevent it from binding its natural ligand;
(ii) binding to the receptors through which Hh signalling is achieved, and preventing, decreasing or attenuating transmission at that receptor;
(iii) down-regulating or de-activating the downstream signalling pathways activated by the modulator binding to the receptors through which Hh signalling is achieved, for example by inhibiting or blocking Smo activity or Gli activity;
(iv) decreasing, preventing or attenuating transcription, translation or expression of the signal transduction molecule Smo;
(v) inhibiting synthesis or release, from intracellular stores, of the signal transduction molecule Smo; and/or
(vi) increasing the rate of degradation of Smo; and/or
(vii) increasing transcription, translation or expression of the receptor Ptch through which Hh signalling is achieved.

It will be appreciated that each of mechanisms (i) to (vii) results in altering transmission at the receptor/signal transduction molecules through which Hh signalling is directed, and the activity thereof, to thereby negatively modulate the Hh signalling. The receptor through which Hh signalling is achieved may be the cell-surface receptor Patched (Ptch), which inhibits activity of the Hh-signal transduction molecule Smoothened (Smo). When Hh binds Ptch, the inhibition of Smo is relieved, and Smo signals into the cell.

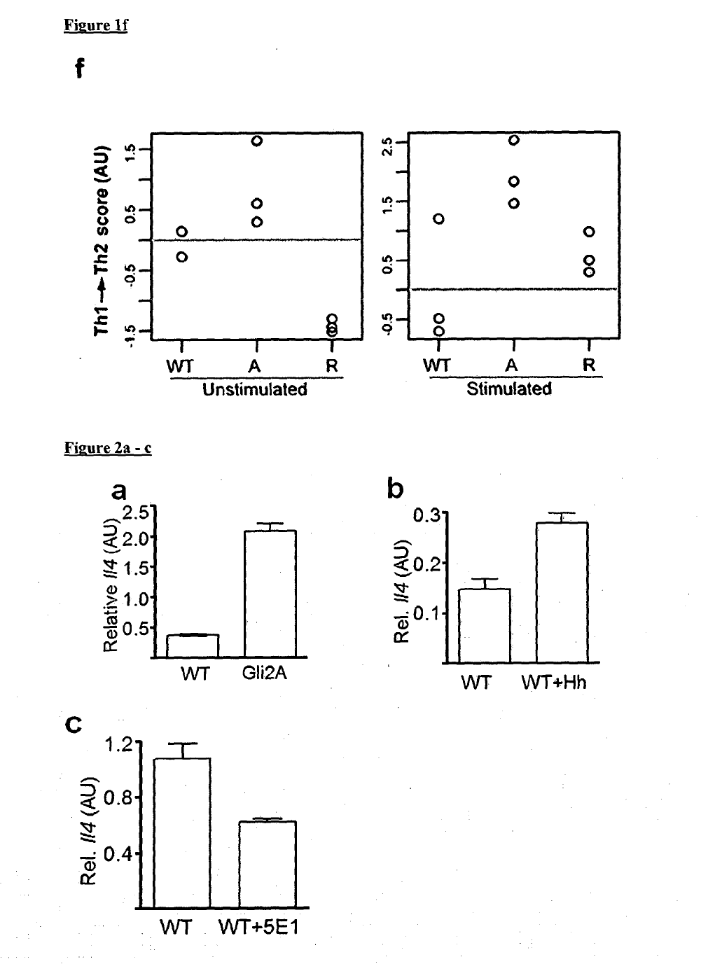

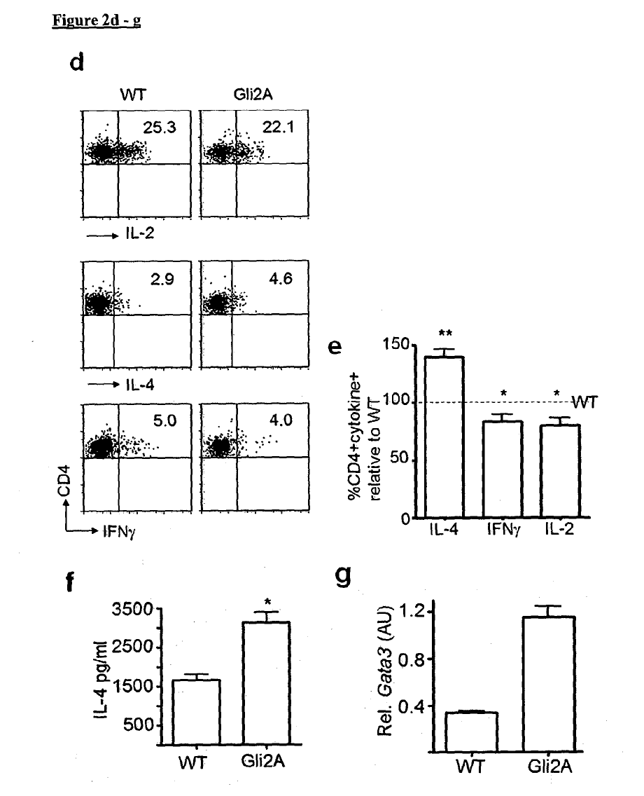

In this embodiment, the modulator may comprise an anti-Hh antibody or an Hh inhibitor, which is capable of altering receptor/signal transduction molecule conformation/stability, or blocking the receptor's activity. The modulator may comprise an anti-Shh, anti-Ihh or anti-Dhh antibody, or a Shh, Ihh or Dhh inhibitor. It will be appreciated that anti-Hh antibodies and suitable Hh inhibitors are well-known to the skilled person. For example, suitable anti-Hh antibodies are commercially available (e.g. anti-Shh from R&D systems, Catalog number: MAB464; anti-Ihh from R&D systems, Catalog number: MAB1705; anti-Dhh from Creative Biomart, Catalog number: 14753MH), and anti-Shh and anti-Ihh antibodies are described in Ericson, J. of al., (1996), Cell 87, 661-673. Examples of suitable Hh inhibitors include cyclopamine (Chen et al, Genes and Development 16, 2743; 2002); and SMO antagonist BMS 833923. One embodiment of an anti-Shh antibody may be that which is known as “5E1”, as used in FIG. 2(c).

Such negative modulators may be used to treat any Th2-mediated disease. The Th2-mediated disease, which may be treated, may be a Th2 inflammatory disease. Examples of Th2-mediated diseases, which may be treated, include cancer, chronic lung disease, asthma, scleroderma, allergy, rhinitis, allergic dermatitis, uticaria, anaphylaxis, atrophy (e.g. muscle) or transplant rejection.

Accordingly, in a preferred embodiment, a negative modulator of Hedgehog (Hh) signalling is used to treat asthma. The negative modulator may be a Sonic Hh (Shh) negative modulator. Advantageously, treatment of asthma using a negative modulator of Hh signalling (e.g. an anti-Hh reagent) may prevent long-term remodeling of the lungs by collagen-deposition. Furthermore, treatment with such modulators will specifically inhibit the cross-talk between lung tissue and lymphocytes, thereby blocking the mechanism of disease. Also, such anti-Hh reagents should cure asthma in some patients with an underlying Hh-related genetic cause. Thus, the anti-Hh therapy according to the invention can be used for stratified, personalized treatment.

As shown in the Examples, the inventors are the first to have established that Hh induces IL4 production, and that resident T-cell responses can be skewed by Hh that is secreted from tumours. Accordingly, based on this observation, it follows that it would be possible to treat Th2-driven cancer by administering a compound which negatively modulates Hh signalling, which promotes a switch away from a Th2 response. Therefore, the cancer which can be treated may be one in which Th2 T-cells are involved in driving the proliferation of the tumour. Thus, the cancer may be Th2-driven cancer. For example, the cancer may be lymphoma, for example B cell lymphoma.

In another embodiment, the modulator may be a positive modulator of Hedgehog (Hh) signalling (for example an agonist), for use in treating a Th1-mediated disease. The positive modulator may be capable of:—
(i) altering the conformational state of the receptors or signal transduction molecules through which Hh signalling is achieved, for example by stabilizing the active conformation of that receptors and/or maintaining the receptors in its active conformation to thereby increase its binding to its natural ligand;
(ii) binding to the receptors through which Hh signalling is achieved, and increasing, promoting or augmenting transmission at that receptors;
(iii) promoting or activating the downstream signalling pathways activated by the modulator binding to the receptor through which Hh signalling is achieved, for example by increasing Smo signal transduction or Gli activity;
(iv) increasing, promoting or augmenting transcription, translation or expression of the signal transducer Smo through which Hh signalling is achieved;
(v) increasing synthesis or release, from intracellular stores, of the signal transducer Smo through which Hh signalling is achieved, or agonists thereof;
(vi) decreasing the rate of degradation of the signal transducer Smo through which Hh signalling is achieved, or agonists thereof; and/or
(vii) decreasing, inhibiting or preventing transcription, translation or expression of the receptor Ptch through which Hh signalling is achieved.

It will be appreciated that each of mechanisms (i) to (vii) results in altering transmission at the receptor/signal transduction complex through which Hh signalling is directed, and the activity thereof, to thereby positively modulate the Hh signalling.

In this embodiment, the modulator may comprise Shh, Ihh or Dhh, or a functional variant or fragment thereof. The modulator may comprise a protein comprising an amino acid sequence substantially as set out in SEQ ID No: 2, 4 or 6, or a functional variant or fragment thereof. The protein may be recombinant, i.e. produced using recombinant DNA technology, known to the skilled person. The protein may be encoded by a nucleic acid sequence substantially as set out in SEQ ID No: 1, 3 or 5, or a functional variant or fragment thereof.

Such positive modulators may be used to treat any Th1-mediated disease. The Th1-mediated disease, which may be treated, may be a Th1 inflammatory disease. Examples of Th1-mediated diseases, which may be treated, include rheumatoid arthritis (RA); psoriatic arthritis; psoriasis; inflammatory bowel syndrome (IBD); Crohn's disease; ulcerative colitis; multiple sclerosis (MS); flu, including pandemic flu; respiratory disorders, for example those caused by viruses, such as respiratory syncytial virus (RSV); cystic fibrosis (CF); herpes, including genital herpes; sepsis and septic shock; bacterial pneumonia; bacterial meningitis; dengue hemorrhagic fever; diabetes Type I; endometriosis; prostatitis; uveitis; uterine ripening; alopecia areata; ankylosing spondylitis; coeliac disease; dermatomyositis; diabetes mellitus Type 1; Goodpasture's syndrome; Graves' disease; Guillain-Barré syndrome; juvenile idiopathic arthritis; Hashimoto's thyroiditis; idiopathic thrombocytopenic purpura; Lupus erythematosus; mixed connective tissue disease; myasthenia gravis; narcolepsy; osteoarthritis; pemphigus vulgaris; pernicious anaemia; polymyositis; primary biliary cirrhosis; relapsing polychondritis; Sjögren's syndrome; temporal arteritis; vasculitis; Wegener's granulumatosis; and age-related macular degeneration.

It will be appreciated that modulators according to the invention may be used in a medicament, which may be used in a monotherapy, i.e. use of only a positive modulator of Hedgehog signalling, which promotes a switch away from a Th1 response, for treating, ameliorating, or preventing a Th1-mediated disease, or the use preventing a Th2-mediated disease, such as asthma or Th2-driven cancer. Alternatively, modulators according to the invention may be used as an adjunct to, or in combination with, known therapies for treating, ameliorating, or preventing Th1- and Th2-mediated diseases, such as asthma or Th2-driven cancer. For example, negative modulators of the invention may be used in combination with known agents for treating asthma, such as steroids or beta-2 agonists. Similarly, positive modulators of the invention may be used in combination with known techniques for treating arthritis.

The modulators according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising modulators according to the invention may be used in a number of ways. For instance, oral administration may be required, in which case the modulators may be contained within a composition that may, for example, be ingested orally in the form of a tablet, capsule or liquid. Compositions comprising modulators of the invention may be administered by inhalation (e.g. intranasally, orally). Compositions may also be formulated for topical use. For instance, creams or ointments may be applied to the skin, for example, adjacent the treatment site. When treating asthma, for example, the composition may be applied to the skin adjacent the lungs.

Modulators according to the invention may also be incorporated within a slow- or delayed-release device. Such devices may, for example, be inserted on or under the skin, and the medicament may be released over weeks or even months. The device may be located at least adjacent the treatment site, e.g. by the lungs. Such devices may be particularly advantageous when long-term treatment with modulators used according to the invention is required and which would normally require frequent administration (e.g. at least daily injection).

In a preferred embodiment, modulators and compositions according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the modulators that is required is determined by its biological activity and bioavailability, which in turn depends on the mode of administration, the physiochemical properties of the modulator and whether it is being used as a monotherapy or in a combined therapy. The frequency of administration will also be influenced by the half-life of the modulators within the subject being treated. Optimal dosages to be administered may be determined by those skilled in the art, and will vary with the particular modulators in use, the strength of the pharmaceutical composition, the mode of administration, and the advancement of the disease being treated. Additional factors depending on the particular subject being treated will result in a need to adjust dosages, including subject age, weight, gender, diet, and time of administration.

Generally, a daily dose of between 0.0 μg/kg of body weight and 0.5 g/kg of body weight of the modulators according to the invention may be used for treating, ameliorating, or preventing the Th1- or Th2-mediated disease, depending upon which modulator is used. More preferably, the daily dose of modulator is between 0.01 mg/kg of body weight and 500 mg/kg of body weight, more preferably between 0.1 mg/kg and 200 mg/kg body weight, and most preferably between approximately 1 mg/kg and 100 mg/kg body weight.

The modulators may be administered before, during or after onset of the Th1- or Th2-mediated disease or Th2-driven cancer. Daily doses may be given as a single administration (e.g. a single daily injection). Alternatively, the modulators may require administration twice or more times during a day. As an example, modulators may be administered as two (or more depending upon the severity of the disease being treated) daily doses of between 25 mg and 7000 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of modulators according to the invention to a patient without the need to administer repeated doses.

Known procedures, such as those conventionally employed by the pharmaceutical industry (e.g. in vivo experimentation, clinical trials, etc.), may be used to form specific formulations comprising the modulators according to the invention and precise therapeutic regimes (such as daily doses of the modulators and the frequency of administration). The inventors believe that they are the first to describe a composition for treating Th1- and Th2-mediated diseases, based on the use of the modulators of the invention.

Hence, in a third aspect of the invention, there is provided a Th1- or Th2-mediated disease treatment composition, comprising a modulator of Hedgehog (Hh) signalling and a pharmaceutically acceptable vehicle.

The term “Th1- or Th2-mediated disease treatment composition” can mean a pharmaceutical formulation used in the therapeutic amelioration, prevention or treatment of any Th1- or Th2-mediated disease in a subject. Examples of such diseases are provided herein. Therefore, the composition may be an asthma or cancer treatment composition.

The invention also provides in a fourth aspect, a process for making the Th1- or Th2-mediated disease treatment composition according to the third aspect, the process comprising contacting a therapeutically effective amount of a modulator of Hedgehog (Hh) signalling and a pharmaceutically acceptable vehicle.

The modulator may comprise a negative modulator of Hh signalling, such as anti-Hh antibody or an Hh inhibitor. Alternatively, the modulator may comprise a positive modulator of Hh signalling, for example Shh, Ihh or Dhh protein, or a functional variant or fragment thereof.

A “subject” may be a vertebrate, mammal, or domestic animal. Hence, compositions and medicaments according to the invention may be used to treat any mammal, for example livestock (e.g. a horse), pets, or may be used in other veterinary applications. Most preferably, however, the subject is a human being.

A “therapeutically effective amount” of the modulator is any amount which, when administered to a subject, is the amount of medicament or drug that is needed to treat the Th1- or Th2-mediated disease, such as asthma or Th2-driven cancer, or produce the desired effect.

For example, the therapeutically effective amount of modulator used may be from about 0.01 mg to about 800 mg, and preferably from about 0.01 mg to about 500 mg. It is preferred that the amount of modulator is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g. the modulator) may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The modulator according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The modulator may be prepared as a sterile solid composition that may be dissolved or suspended at the time of administration using sterile water, saline, or other appropriate sterile injectable medium.

The modulators and pharmaceutical compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The modulators according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

The inventors have observed that the ability to increase Th2 differentiation using positive modulators of Hh signalling can be effectively harnessed in the design and application of vaccines. It will be appreciated that a vaccine comprises T-cell and B-cell epitopes, which induce humoral immunity in a vaccinated subject. Therefore, a positive modulator of Hh signalling (e.g. a Hh protein) may be added to the vaccine in order to increase Th2 differentiation, and thereby increase the ability of the subject's T-cells to help the B-cells to produce antibodies upon administration of the vaccine to a subject.

Thus, according to a fifth aspect of the invention, there is provided an adjuvant comprising a positive modulator of Hedgehog (Hh) signalling.

According to a sixth aspect, there is provided use of a positive modulator of Hedgehog (Hh) signalling, as an adjuvant.

The positive modulator may be as defined above. The modulator may comprise Shh, Ihh or Dhh, or a functional variant or fragment thereof. The modulator may comprise a protein comprising an amino acid sequence substantially as set out in SEQ ID No: 2, 4 or 6, or a functional variant or fragment thereof. The protein may be encoded by a nucleic acid sequence substantially as set out in SEQ ID No: 1, 3 or 5, or a functional variant or fragment thereof.

According to a seventh aspect, there is provided a vaccine comprising the adjuvant of the fifth aspect.

It will be appreciated that an adjuvant is a pharmacological or immunological agent, which modifies the effect of other active agents in the vaccine (e.g. the epitopes), while having few, if any, direct effects when administered by itself. Adjuvants are frequently included in vaccines to enhance the recipient's immune response to an administered antigen or immunogen, while keeping the administered foreign material to a minimum. Although such immunological adjuvants have traditionally been viewed as substances that aid the immune response to an antigen or immunogen, adjuvants have also evolved as substances that can aid in stabilising formulations of antigens, especially vaccines administered for animal health.

The adjuvant may be used in a vaccine comprising an immunogen, wherein the immunomodulatory activity of the immunogen in the presence of the adjuvant is greater than its immunomodulatory activity in the absence of the adjuvant. Thus, the adjuvant of the fifth aspect may be immunostimulatory. Advantageously, the adjuvant may be capable of enhancing the immunomodulatory activity of a subject administered with the adjuvant, resulting in the stimulation of the immune system, for treating hypo-immune conditions, such as cancer and immuno-suppression.

According to a eighth aspect, there is provided the vaccine according to the seventh aspect, for use in therapy.

According to a ninth aspect, there is provided the vaccine according to the seventh aspect, for use in vaccination.

According to a tenth aspect, there is provided a method of eliciting, in a subject, an effective immune response, the method comprising administering, to a subject, an effective amount of the vaccine of the seventh aspect.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. The terms “substantially the amino acid/nucleotide/peptide sequence”, “functional variant” and “functional fragment”, can be a sequence that has at least 40% sequence identity with the amino acid/nucleotide/peptide sequences of any one of the sequences referred to herein, for example 40% identity with the nucleotide sequence identified as SEQ ID No:5 (i.e. Dhh cDNA) or the protein identified as SEQ ID No:6 (i.e. Dhh protein), or 40% identity with the nucleotide identified as SEQ ID No: 1 (i.e. Shh gene) or the protein identified as SEQ ID No:2 (i.e. Shh protein), and so on.

Amino acid/polynucleotide/polypeptide sequences with a sequence identity which is greater than 50%, more preferably greater than 65%, 70%, 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90%, 92%, 95%, 97%, 98%, and most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to the sequences shown in SEQ ID No's: 1, 3 or 5 or their complements under stringent conditions. By stringent conditions, we mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in SEQ ID No:2, 4 or 6.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

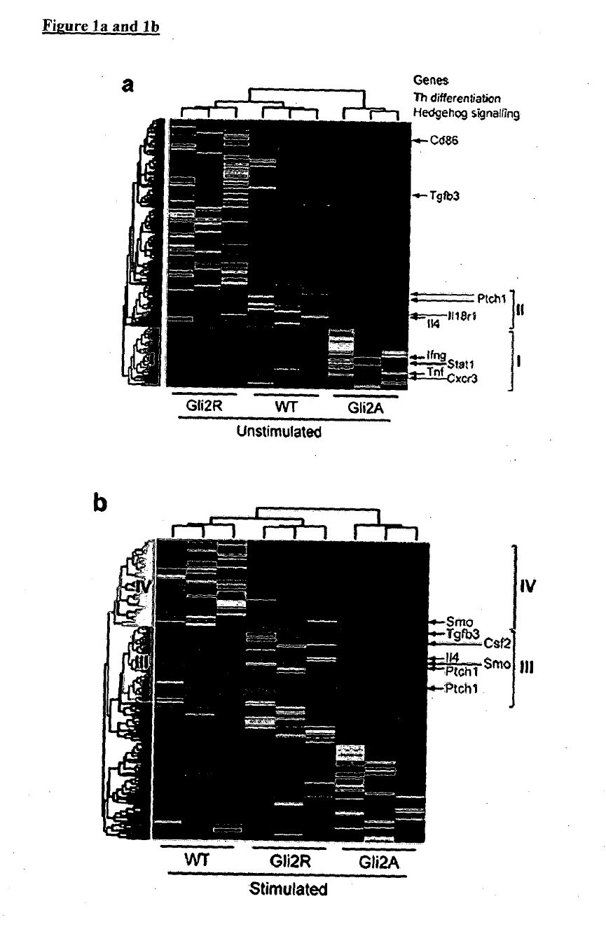

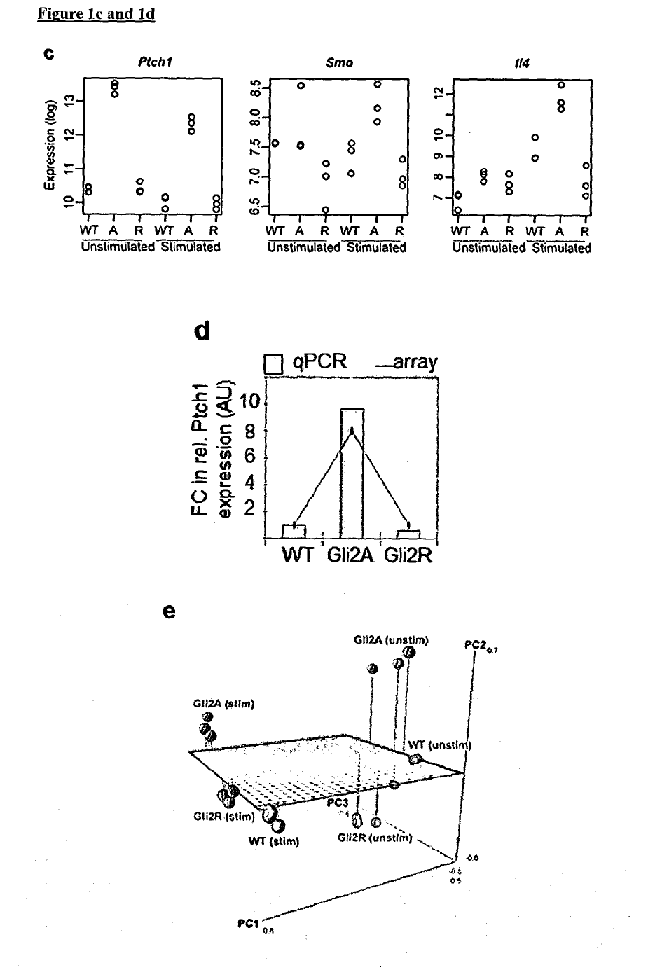

FIG. 1 shows that Hedgehog-dependent transcription of genes are involved in CD4+ T-cell differentiation; (a, b) Heatmaps showing expression of significant genes in array data sets for (a) unstimulated and (b) stimulated CD4+ T-cells from WT, Gli2A and Gli2R spleen. Selected genes for Th differentiation and Hh signalling are indicated. (c) Expression of Hh-signalling genes (Ptch1 and Smo) and of Il4 from array data. (d) qPCR (n=3, in triplicate) for Ptch1 relative to Hprt, normalised to WT samples on RNA from microarray experiments to compare fold-changes in mean expression generated by Affymetrix and qPCR. (e) 3D-PCA showing sample relationships in PC1, PC2 and PC3. (f) Samples were measured by Th1→Th2 score, where 0 is the mean value of WT samples. Relative skewedness to gene expression profiles in comparison to other publically-available microarray datasets from Th1 (negative) or Th2 (positive) samples is shown, AU: arbitrary units;

FIG. 2 shows that Hedgehog-dependent transcription alters Th1/Th2 cytokine production; (a) Purified CD4+WT and Gli2A splenocytes stimulated for 48 h with anti-CD3/CD28-coated beads, or (b) WT CD4+ cells stimulated and cultured with rShh or (c) anti-Shh (5E1) for 48 h. Example qPCR performed in triplicate on cDNA to quantify expression of 114 relative to Hprt (AU: arbitrary units). Mean Il4 expression per independent sample was calculated relative to WT (unpaired t tests: A, n=3 p=0.04; B, n=7 p=0.03; C, n=4 p=0.02). (d) Fresh splenocytes subject to ic-cytokine staining to quantify expression of IL-2, IL-4 and IFNγ. (e) Mean percentage expression of cytokine in Gli2A cells expressed relative to WT levels shown for 6 mice of each genotype (unpaired t tests: IL-4, p=0.0001; IFNγ, p=0.03; IL-2, p=0.007). (f) Mean production of IL-4 measured by ELISA (WT n=2, Gli2A n=3, unpaired t test, p=0.02). (g) Purified CD4+ cells activated for 48 h with anti-CD3/CD28-coated beads. Expression of Gata3 relative to Hprt, quantified by qPCR as described;

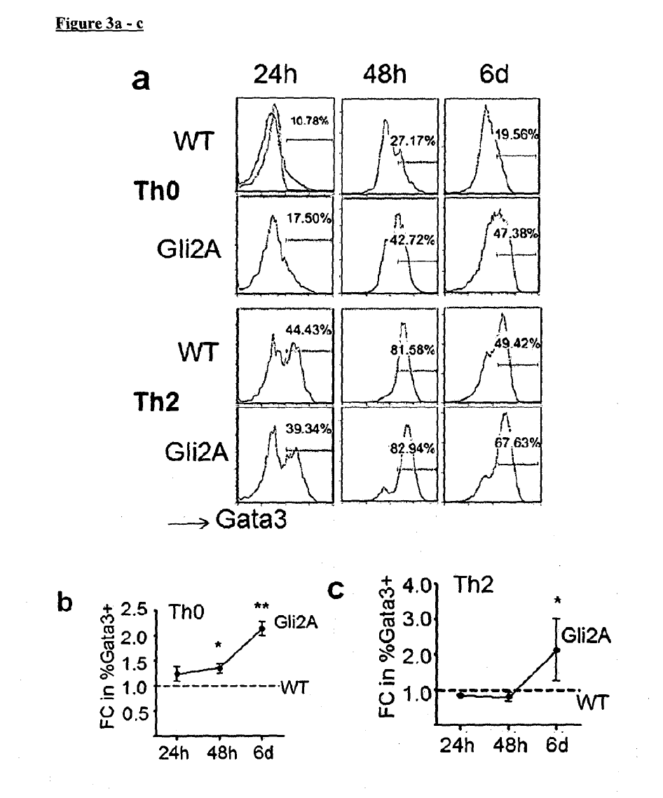

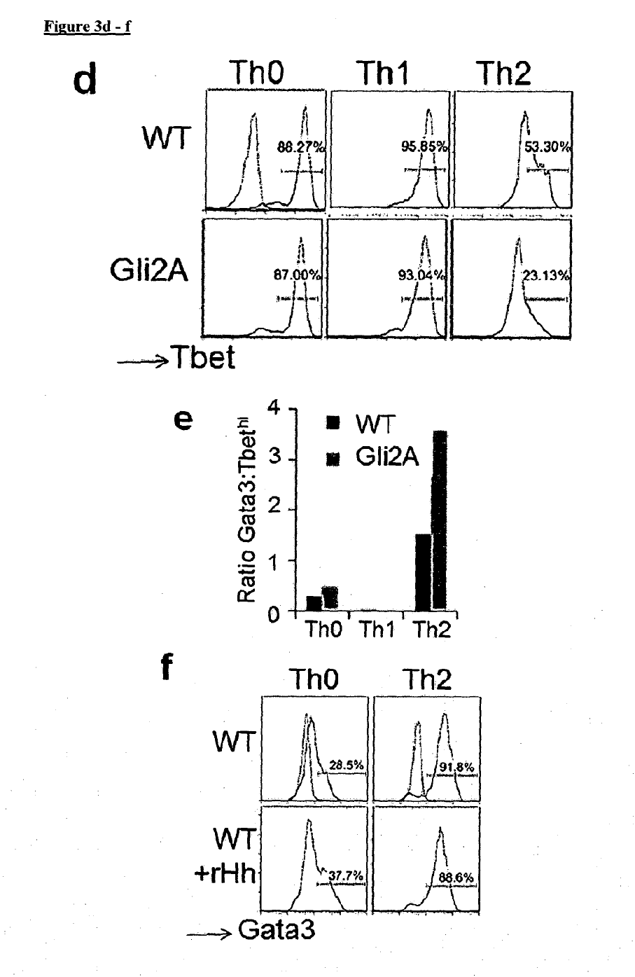

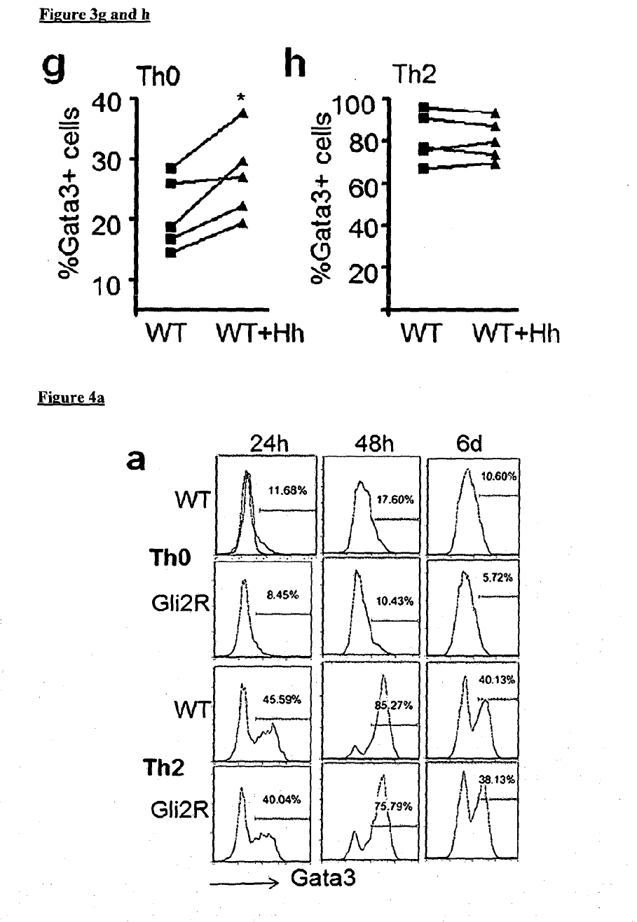

FIG. 3 shows that active Hh-dependent transcription upregulates expression of Gata3 and skews cells towards a Th2 phenotype in vitro; (a) Purified CD4+ cells from WT and Gli2A spleen were cultured for 6 d in Th skewing conditions as described. Gata3 protein expression was measured by ic-staining and flow cytometry. (b, c) Mean percentage of Gata3+ cells, calculated as fold change (FC) of the WT cultures (WT set to 1) for three independent experiments in (b) Th0 (48 h & 6 d, unpaired t test, *p=0.01, **p=0.001) and (c) Th2 conditions (24 h & 6 d, unpaired t test, p=0.02). (d) Ic-Tbet protein, quantified by flow cytometry in Th0, Th1 and Th2 conditions. (e) Example Gata3:Tbet ratio calculated at 48 h. (f) WT CD4+ cells cultured for three days with or without rShh in (g) Th0 (paired t test on % Gata3+, n=5: p=0.04) and (h) Th2 conditions (n=5). Example overlays of ic-isotype control antibody staining shown as dotted histograms in a, d & f;

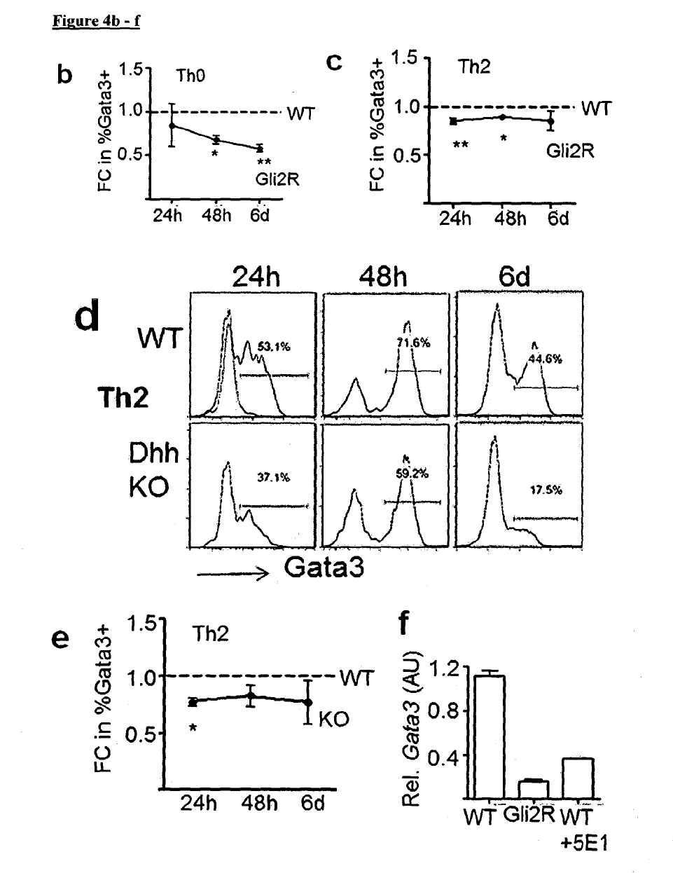

FIG. 4 shows that physiological Hh-signalling controls expression of Gata3 and Il4; (a) Gata3 protein expression in purified WT and Gli2A CD4+ splenocytes. (b, c) Mean percentage Gata3+ cells was calculated as fold change (FC) that of the WT cultures for three independent experiments in (b) Th0 (48 h & 6 d, unpaired t test: *p=0.004, **p=0.009) and (c) Th2 conditions (24 h & 48 h, unpaired t test: **p=0.005, *p<0.05). (d, e) Gata3 protein expression in CD4 cells from Dhh KO and WT littermates (e, n=3 experiments) cultured in Th2 conditions (24 h unpaired t test: p=0.0004). Example overlays of isotype control antibody staining shown as a dotted histogram in the upper left panel of A & D. (f) Mean Gata3 mRNA expression by qPCR relative to Hprt, representative of three independent experiments. CD4+ cells from Gli2R, WT, or WT spleen treated with anti-Hh (5E1) were cultured for 48 h with anti-CD3/CD28-coated beads. Mean Gata3 expression was calculated relative to untreated WT groups (p<0.01). (g) Schematic of the murine Il4 locus indicating the HS2 region and primers used to amplify region containing predicted Gli binding site (not to scale). (h, i) ChIP-PCR performed on sonicated chromatin from Gli2A CD4+ cells after 48 h anti-CD3/28 stimulation using antibodies specific to Gli2 and control mouse IgG (mIgG) and RNA Pol II (Pol). Immunoprecipitated DNA was amplified by PCR from the HS2 region in intron 2 of Il4, and analysed by qPCR for fold enrichment in the Gli2-immunoprecipitated fraction based on the input fraction and negative control;

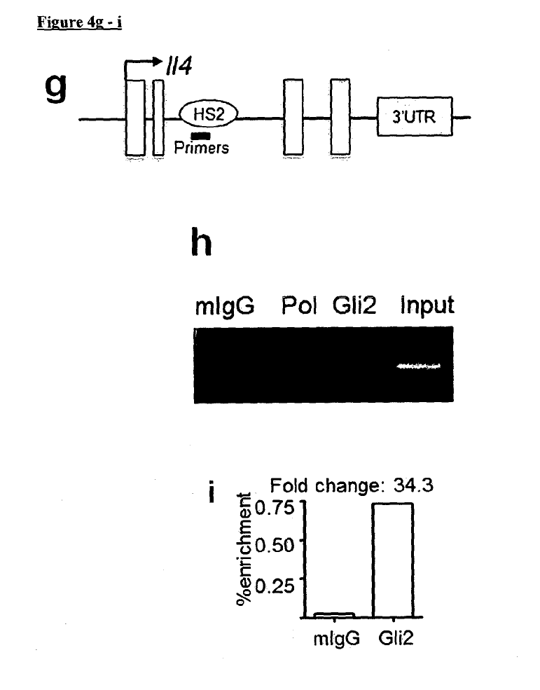

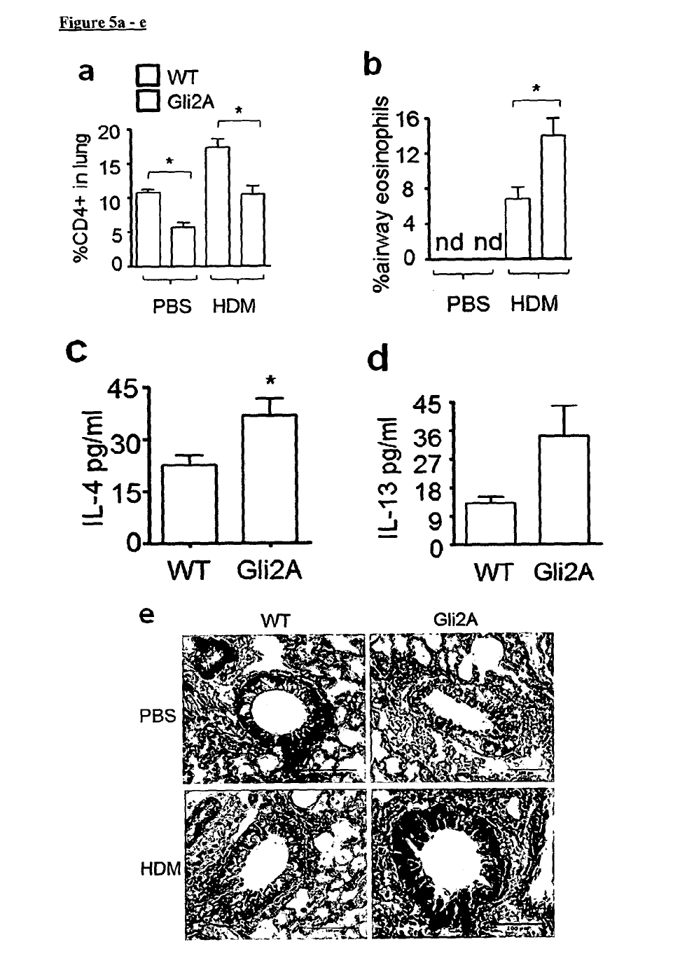

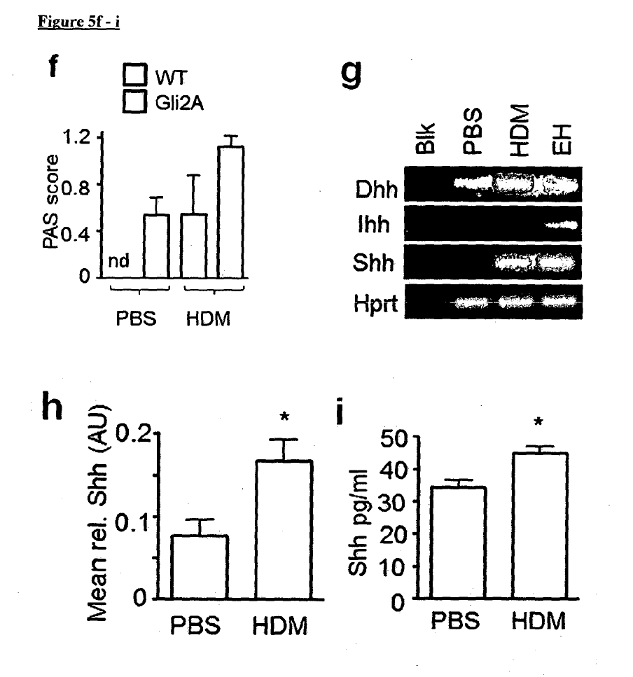

FIG. 5 shows that active Hh-dependent transcription enhances Th2-mediated disease in allergen-challenge allergic airways disease; WT and Gli2A (n=5 in each group) cells from lung, airway and draining LN were analysed after three-times weekly intranasal challenge with PBS or 15 μg purified HDM extract allergen. (a) Flow cytometric analysis of % CD4+ T-cells present in lung (3 wks) and (b) % eosinophils (CD11b+ SiglecF+) in BAL (3 wks). (c, d) ELISA analysis (2 wks) to quantify IL-4 (unpaired t test: p=0.04) and IL-13 produced (p=0.08). (e, f) Lung lobes analysed using periodic acid shift (PAS) by histological scoring (bar indicates 100 μm) to assess bronchioles for mucus-secreting goblet cells (non-parametric ANOVA: p=0.04). nd: not detectable. (g) Expression of Dhh, Ihh and Shh by non-quantitative RT-PCR in lung tissue from PBS- or HDM-treated WT BALB/c mice; Blk: water control; EH: embryo head positive control (h) mean expression of Shh relative to Hprt (p=0.02) by qPCR and (i) quantification of Shh protein by ELISA (p=0.04) in homogenised lung of WT BALB/c mice treated with PBS (n=4) or HDM (n=6) as described; and

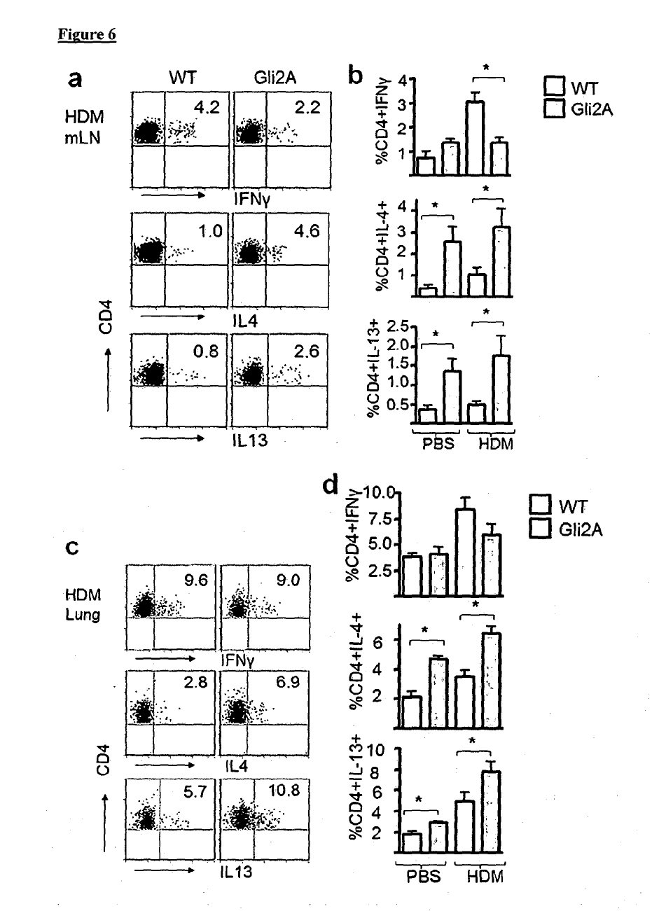

FIG. 6 shows that Hh-dependent transcription enhances production of Th2 cytokines by T cells in draining LN and lung tissue during the allergen-challenge airways disease model; WT and Gli2A mice (n=5 in each group) underwent repeated intranasal challenge with PBS or HDM allergen as described. Ic-cytokine staining in CD4+ cells from (a, b) mLN and (c, d). Statistically significant differences between groups were compared by unpaired t testing and are indicated by bars (*p<0.05).

### EXAMPLES

### Materials and Methods

### Mice

Animal experiments were performed with Lck-Gli2ΔN2 mice (Rowbotham, N. J. et al. Blood 109, 3757-3766, 2007), Lck-Gli2ΔC2 mice (Rowbotham, N. J. et al. Cell Cycle 7, 904-908, 2008), and Dhh KO mice (Bitgood, M. J. et al., Current biology: CB 6, 298-304, 1996) and littermate or age-matched controls, under UK Home Office ethics and regulations. The allergic airways disease model was as described (Gregory, L. G. et al., Clin Exp Allergy 39, 1597-1610, 2009).

### Flow Cytometry

Cells were stained using antibodies and intracellular (Ic)-cytokine/transcription factor staining kits (BD Pharmingen or eBiosciences). Samples were acquired on FACScan, FACScalibur or LSRII flow cytometers (BD) and analysed using FlowJo (Tree Star).

### CD4+ T-Cell Purification

Splenocytes were magnetically purified using the Easy Sep mouse CD4+ cell negative selection kit (StemCell Technologies).

### Cell Culture

T-cells were cultured at 5×10⁶/ml in AIMV (Invitrogen) with 10⁻⁵ M β-mercaptoethanol (Sigma-Aldrich). For microarray cells were activated for 6 h with 0.01 μg/ml soluble anti-CD3 and anti-CD28 (BD). For ChIP and qPCR experiments, CD4+ cells were cultured for 48 h with anti-CD3/anti-CD28 coated beads (1:1 ratio, Invitrogen). Where stated, 500 ng/ml rmShh (R&D systems) or 5 μg/ml 5E1 (anti-Hh mAb, DSHB, Iowa) were used. For Ic-cytokine staining, splenocytes were cultured for 3-4 hours with 50 ng/ml PMA (Sigma), 500 ng/ml Ionomycin (Sigma) and 3 μg/ml Brefeldin A (eBiosciences).

For Th skewing cultures, CD4+ cells were cultured in complete RPMI+FCS (Invitrogen) for 6 days. For Th0 neutral conditions, 5×10⁵/ml cells were cultured on 5 μg/ml plate-bound anti-CD3 with 1 μg/ml soluble anti-CD28. For Th1 conditions, 5 ng/ml rmIL-12 and 5 μg/ml anti-mouse IL-4 were added. For Th2 conditions, 10 ng/ml rmIL-4, 5 μg/ml anti-mouse IFNγ, 5 μg/ml anti-mouse IL-12 was added (antibodies/proteins: eBioscience).

### ELISA

IL-4 concentration in culture supernatants was analysed using the Th1/Th2 panel ELISA kit (eBioscience). For BAL, ELISA were performed using the Ready-Set-Go kits for IL-4 and IL-13 (eBioscience). Shh ELISA was performed using the R&D systems Mouse Shh DuoSet ELISA kit.

### Histology

Fixed lung sections were analysed using the Periodic acid Schiff stain. Sections were scored double-blind for airways demonstrating positive staining.

### Microarray and Data Analysis

Total RNA was extracted from CD4+ splenocytes using Agilent's Absolutely RNA kit. UCL Genomics processed total RNA for hybridisation to Affymetrix MOE430 2.0 mouse whole genome array chips. Data were acquired according to standard Affymetrix protocols and deposited in the GEO repository (GSE33156, available Jan. 9, 2012). Microarray data were normalised using mas5 of affy in each dataset. Differentially expressed genes (DEG) were identified by p<0.05 considering a false discovery rate by limma (Bioconductor). Unstimulated datasets showed 805 DEG, stimulated showed 368 DEG. Heatmaps were generated using the heatmap.2 function gplots (Bioconductor). Hierarchical clustering was performed on both genes and samples according to Euclidean distance using the complete agglomeration method. Th differentiation genes were identified using the gene list from ‘Mouse Th1/Th2/Th3 PCR array’ (SABiosciences).

Visualisation of three-dimensional sample relationships by PCA (3D-PCA) was generated by 3dscatter (CRAN package, car). PCA was performed using dudi.pca (CRAN, ade4). 3D-PCA has been previously used for estimating sample similarities (Bushel, P. R. et al. Proc Natl Acad Sci USA 104, 18211-18216, 2007). To assess features of the array samples in terms of a Th1→Th2 axis, we employed the GEO dataset GSE14308, which analysed in vitro differentiated Th1 and Th2 cells from primary mouse CD4 cells using Affymetrix arrays. In order to compare our dataset with the external data, we employed a novel application of canonical correspondence analysis (Greenacre, M. J. Correspondence analysis in practice. 2nd edn, (Chapman & Hall/CRC, 2007), where a Th1-Th2 axis was extracted from GSE14308 using PCA, and used as a gradient for canonical correspondence analysis of our own datasets.

Quantitative Real Time RT-PCR (qPCR)

Sample preparation and qPCR were as described (Hager-Theodorides, A. L. et al. J Immunol 183, 3023-3032, 2009). Primers were purchased as pre-validated oligonucleotides (Quantitect Assays, Qiagen) except: Ptch Ex14F TGCTCTCCCAGTTCTCAGACTC (SEQ ID No:7), PtchR Ex14/15R CCACAACCTTGGCTTTMG (SEQ ID No:8); All samples were analysed in triplicate and expressed as mean±SD following normalisation to expression of Hprt, and independently verified in two or three separate experiments.

### Chromatin Immunoprecipitation

ChIP was performed using the ChampionChIP kit (SABiosciences, Qiagen). Briefly, 1×10⁷ stimulated CD4+ cells were fixed, lysed and chromatin was sonicated to 500-1000 bp fragments using the Bioruptor Sonicator (Diagenode). This was pre-cleared and immunoprecipitated with anti-Gli2 (Santa Cruz), anti-RNA polymerase II (SABiosciences) or mIgG (SABiosciences). DNA was purified and used in PCR/qPCR using primers specific for HS2 region of Il4 (primer-pair21-Tanaka, S. et al. Nat Immunol 12, 77-85, 2011) and ChampionChIP Gata3 primer assays (SABiosciences). Results were validated in replicate experiments.

### Data Analysis

Statistical analyses were performed using Microsoft Excel or Prism 4 (Graph Pad) using Student's two-tailed unpaired or paired t-tests. For PAS scores, non-parametric ANOVA was used. Significance was reached at p<0.05.

### Example 1

### Gli2 Modulates the Expression of Many Genes in Resting and Activated T-Cells

In order to define the transcriptional response of CD4+ T-cells to Hh pathway activation, the inventors examined Hh-dependent gene expression in resting and activated T-cells.

The inventors have established transgenic models where transcription by Gli2 is either constitutively activated or repressed in T-lineage cells. Gli2 has an N-terminal repressor domain and a C-terminal activator domain (Sasaki, H. et al., Development 126, 3915-3924, 1999). Lck-Gli2ΔN2 (Gli2A) mice carry a transgene encoding a truncated form of Gli2 that acts as a permanent transcriptional activator of Hh target genes (Rowbotham et al, 2007). Conversely, Lck-Gli2ΔC2 (Gli2R) mice express a repressor of Gli2-dependent transcription, which by binding to Gli-binding sites inhibits endogenous Hh-dependent transcription, and hence Hh signal transduction, in the cell (Rowbotham et al, 2008). Thus, comparison of transcriptional profiles in WT and Gli2R cells would identify genes whose expression is regulated by Hh signal transduction under physiological conditions.

The inventors performed Affymetrix whole genome array analysis on CD4+ T cells from WT, Gli2A and Gli2R spleen. RNA was extracted from purified fresh, resting CD4+ cells (unstimulated) and from CD4+ cells stimulated with anti-CD3/CD28 for 6 h (stimulated) to obtain transcriptional profiles before and during the early stages of T-cell activation (GEO ref: GSE33156). Hundreds of differentially expressed genes (DEG) were identified between WT and transgenic groups, indicating that Hh-dependent transcription has wide-ranging effects on T-cells. Samples clustered according to genotype (FIG. 1a: unstimulated; b: stimulated) and Hh signalling/responsive genes, Ptch1 and Smo, were upregulated in Gli2A but not Gli2R (FIG. 1a, cluster II and FIG. 1B, clusters III and IV) as expected. Surprisingly, genes involved in Th-cell differentiation were differentially expressed between Gli2A, Gli2R and WT (FIG. 1a, b). The Th2 cytokine, Il4, was upregulated in Gli2A and belonged to the same cluster as Ptch1, a known Hh target gene, in unstimulated (FIG. 1a, cluster II & FIG. 1c) and stimulated (FIG. 1b, cluster III & FIG. 1c) samples. This suggests that Il4 was downstream of Hh signalling in T-cells. In contrast, Th1-related genes including Ifng, Tnf, Stat1 and Cxcr3 were downregulated in unstimulated Gli2A and upregulated in unstimulated Gli2R cells (FIG. 1a, cluster I). As expected, the known Hh target gene, Ptch1 was strongly upregulated in Gli2A compared to WT and Gli2R (FIG. 1c).

In order to validate the microarray study, the inventors performed qPCR on genes of interest in WT, Gli2A and Gli2R CD4+ T-cells using RNA from microarray experiments. The inventors found that mean relative expression of Ptch1 was upregulated approximately nine-fold in Gli2A cells vs. WT, in agreement with the data obtained from the microarray (FIG. 1d). Ptch1 was downregulated two-fold in Gli2R cells compared to WT, indicating that active Hh signalling is taking place in T cells ex vivo. Fold expression change correlated well between microarray and qPCR analysis. The inventors also confirmed expression patterns of genes of interest in CD4+ cells from independent sorts by qPCR (data not shown).

T-cell activation causes dramatic changes in transcriptomes, complicating simultaneous comparisons and visualisation of gene expression profiles in unstimulated and stimulated cells by heatmap analysis. Therefore, the inventors applied principle component analysis (PCA) in order to identify common effects of Hh signalling in resting and activated T-cells (FIG. 1e).

Principle component 1 (PC1) showed that the largest difference was between the unstimulated and stimulated samples, indicating that activation stimuli caused profound changes in transcription. PC2, the second largest measure of difference in gene expression, reflected differences between Gli2A and the other samples, especially Gli2R (FIG. 1e). Hh signalling/responsive genes, Ptch1 and Smo, and Il4 had high PC2 scores, indicating strong association with Gli2A, and therefore active Hh signalling. However, Th1-related genes showed negative PC2 scores, suggesting that expression of these genes was increased in Gli2R and suppressed in Gli2A, confirming the results in FIGS. 1a and b (data not shown).

### Example 2

### Gli2A Skews CD4 T Cells Towards a Gene Expression Profile Similar to Th2 Cells

These analyses suggested that Gli2A cells are transcriptionally skewed towards Th2. To test this, the inventors used a method with minimal assumptions to generate a scale of Th1/Th2 skewedness (Th1→Th2 score) based on publicly available whole-genome array data derived from Th-skewed cells (GEO database ref: GSE14308). The inventors found Gli2A samples showed high, Th2-like scores when cells were either resting or stimulated (FIG. 1f), indicating Gli2A cells are more Th2-biased compared to other samples. Although unstimulated Gli2R had a low score, suggesting a Th1 bias, this trend disappeared upon stimulation (FIG. 1f). Together, these analyses suggested that Hh signalling in T-cells mediated transcriptional changes that promote Th2 differentiation. The inventors therefore proceeded to test this hypothesis experimentally in vitro and in vivo.

### Example 2

### Gli2-Dependent Transcription Alters T-Cell Cytokine Profiles

As Gli2A cells displayed a transcriptional profile similar to Th2 cells, the inventors investigated the abundance of Il4 transcript by qPCR in activated CD4+ T-cells from WT and Gli2A spleen. After 48 h stimulation with anti-CD3/CD28-coated beads, Il4 expression in Gli2A cells was upregulated ˜6-fold that of WT (FIG. 2a). Expression of Il4 was also upregulated by activated WT cells cultured with recombinant Shh (rShh), confirming that this effect is Hh-specific (FIG. 2b), and was repressed by the addition of neutralising anti-Hh mAb (5E1) to the cultures (FIG. 2c). Thus, physiological Hh signalling increases Il4 transcription in WT T-cells.

In order to examine induction of cytokine production by fresh CD4+ T-cells, the inventors measured intracellular cytokines in WT and Gli2A splenocytes. Interestingly, Gli2A cells showed significantly higher levels of intracellular IL-4 (FIG. 2d, e) and reduced levels of IFNγ (FIG. 2d, e) relative to WT. The inventors also found decreased levels of intracellular IL-2 in Gli2A cells compared to WT (FIG. 2d, e), explaining the previous observation that exogenous IL-2 restored their proliferation to WT levels on CD3/CD28 ligation. To confirm that Gli2A T-cells produce more IL-4 protein, the inventors stimulated lymphocytes in vitro with anti-CD3 and assayed IL-4 secretion by ELISA. Gli2A cells produced on average twice as much IL-4 as WT (FIG. 2f).

The Th2 lineage-specific transcription factor Gata3 was not upregulated in Gli2A cells at 6 h (microarray data). However, as Gli2A CD4+ cells upregulated Il4 mRNA expression and cytokine production upon stimulation, the inventors tested whether Gata3 expression was increased in these cells after 48 h activation. qPCR analysis showed that the presence of Gli2A led to a greater induction of Gata3 than in WT stimulated cells (FIG. 2g).

### Example 4

### Activation of Hh-Dependent Transcription Favours Differentiation to Th2

The relationship between Il4 and Gata3 induction and the initiation of Th2 differentiation is not fully understood, although both proteins are required, and once activated, become co-regulatory. Expression of Gata3 can be induced and maintained by Th2-skewing conditions, occurs over a course of days (Ouyang, W. et al. Immunity 9, 745-755, 1998), and can be used as a measure of Th2 identity. The inventors hypothesised that the propensity of Gli2A cells to upregulate both Il4 and Gata3 after TCR stimulation would influence the early stages of Th differentiation in vitro. Therefore, to test if Hh-dependent transcription influenced Th-differentiation, the inventors cultured WT and Gli2A CD4+ cells in Th skewing conditions following activation, and measured commitment to Th2, by intracellular expression of Gata3 protein.

In neutral Th0 conditions, levels of Gata3 were always higher in Gli2A compared to WT CD4+ cells (FIG. 3a, b), suggesting an inherent bias towards Th2. In Th2 conditions, where exogenous IL-4 is added, WT cells displayed Gata3 levels similar to Gli2A cells at 24 h and 48 h, suggesting that enhanced expression of Gata3 by Th0-conditioned Gli2A cells is the result of their increased IL-4 production. However, after six days in culture, on average double the proportion of Gli2A cells expressed Gata3 than WT, showing enhanced commitment to the Th2 lineage (FIG. 3a, c). Th1 differentiation is controlled by Tbet, which antagonises the effects of Gata3 and vice versa (Chakir, H. et al., J Immunol Methods 278, 157-169, 2003). To test whether Gli2A cells displayed decreased Th1 potential compared to WT, the inventors examined Tbet expression by staining in skewing cultures at 48 h. Expression of Tbet was similar in Th0- or Th1-conditioned cultures, but in Th2 conditions was decreased in Gli2A cells compared to WT (FIG. 3d). This resulted in an increased ratio of Gata3:Tbet, particularly in Th2-skewed Gli2A CD4+ cultures (FIG. 3e). As these two transcription factors are mutual antagonists, and it is the balance of expression of each that determines outcome, the high ratio of Gata3:Tbet in the Gli2A TCR-stimulated CD4 cells compared to WT confirms that these cells are biased to Th2 differentiation.

The inventors then cultured WT CD4+ cells in neutral Th0 conditions for 72 h with a single dose of rShh. By 48 h, Gata3 expression was higher in Hh-treated cells in Th0 conditions, verifying that Hh signalling skews differentiation to Th2 in WT cells (FIG. 4f, g). To ask if the increase in Gata3 expression on rShh treatment was the result of increased IL-4 transcription, the inventors cultured WT CD4+ cells in Th2 conditions in the presence of rShh. As expected, when IL-4 was added to the cultures, Gata3 expression was not affected by rShh (FIG. 4h).

### Example 5

### Reduction of Physiological Hh Signalling in T-Cells Impairs Th2 Differentiation

To test whether repression of physiological Hh-dependent transcription would impair Th2 potential and suppress Gata3 expression, the inventors performed in vitro skewing experiments using Gli2R CD4+ cells. In Gli2R cells, Gata3 did not reach WT levels over the time course in Th0 (FIG. 4b) or Th2 (FIG. 4c) conditions, indicating that repression of physiological Hh pathway activation in T-cells reduces their ability to differentiate to the Th2-lineage. The fact that addition of exogenous IL-4 and anti-IFNγ (in Th2 conditions) to Gli2R cultures was not sufficient to restore Gata3 expression to WT levels indicates that other factors in addition to these cytokines are involved in the Hh-mediated modulation of Th differentiation.

These data indicate that physiological levels of Hh pathway activation in T-cells isolated fresh from the mouse spleen skews transcriptional processes to favour Th2 differentiation. Given this, the inventors examined the effect of reducing environmental Hh protein in the spleen on Th2 differentiation of non-transgenic CD4+ cells ex vivo. The inventors used Dhh^{-/-} knockout (KO) CD4 splenocytes, as Dhh is expressed by spleen stroma (Perry, J. M. et al. Blood 113, 911-918, 2009), and unlike Shh^{-/-}, is not an embryonic lethal mutation. The inventors cultured Dhh KO and WT cells in Th2 skewing conditions and found that Dhh KO cells upregulate Gata3 less efficiently than WT after stimulation (FIG. 4d). This was most pronounced at 24 h (FIG. 4e), suggesting that fresh KO cells are inherently impaired in their ability to undergo rapid Th2 differentiation, as a result of reduction in Hh signal from their environment.

Given that Gata3 protein expression was lowered by a reduction in Hh signalling, the inventors measured Gata3 mRNA in Gli2R CD4 cells and in WT cells treated with anti-Hh mAb after 48 h anti-CD3/CD28 stimulation. Gata3 transcription was reduced by inhibition of Hh signalling (FIG. 4f). Taken together, these experiments show that the physiological Hh signal functionally controls expression of this lineage-specifying transcription factor in CD4+ splenocytes.

### Example 6

### Gli2 Binds Directly to an Intronic Enhancer Region in the Murine Il4 Gene

The rapid (6 h) induction of Il4 in Gli2A cells (FIG. 1c), increased IL-4 production (FIG. 2), altered Gata3 induction (FIGS. 3, 4) and marked repression of Gata3 expression in Gli2R CD4+ T-cells and 5E1-treated WT cells (FIG. 4) prompted us to investigate whether Gli2 could be acting directly to initiate transcription of Il4 and/or Gata3. The inventors examined genomic sequences for suggested Gli consensus binding sequences and found several potential sites in both Gata3 and Il4 genomic sequences. The inventors therefore investigated whether Gli2 can directly bind Il4 and Gata3 by chromatin immunoprecipitation (ChIP). CD4+ cells from Gli2A mice were stimulated for 48 h and then fragmented chromatin was immunoprecipitated with anti-Gli2 or control antibodies. DNA was purified from bound targets and PCR was performed to amplify regions of the Gata3 and Il4 genes identified as containing potential Gli binding sites. The inventors found no enrichment of Gata3 by conventional PCR or qPCR, and so the inventors found no evidence that Gli proteins interact directly with this gene (data not shown). However, in the case of the Il4 locus (FIG. 4g), the inventors observed significant binding to a region localising to an enhancer of Il4 located in intron 2 of the gene (FIG. 4h, i), showing that Gli2 can directly interact with Il4 at a key regulatory region. These data together therefore identify Il4 as a novel target gene of Hh signalling and provide a mechanism for the role of Gli2 in skewing Th differentiation.

### Example 7

### Active Hh-Dependent Transcription Enhances Th2-Associated Pathology in the Murine Model of Allergic Airways Disease

The in vitro data show that activation of Hh signalling predisposes T-cells to become Th2-like via enhanced activation of Il4 transcription by Gli proteins. To test whether Hh-dependent transcription controls Th differentiation and function in vivo, the inventors used a well-established murine model of allergic airways disease, where dosing with house dust mite (HDM) extract elicits a Th2 response (Gregory, L. G. et al. Clin Exp Allergy 39, 1597-1610, 2009). After repeated intranasal administration of allergen or PBS bronchoalveolar lavage fluid (BAL), lung lobes and mediastinal lymph nodes (mLN) were collected from WT and Gli2A animals. The percentage of CD4+ cells infiltrating lung was reduced in Gli2A mice compared to WT (FIG. 5a) regardless of treatment, reflecting the decreased proportion of peripheral T-cells in these mice (Rowbotham, 2007). For WT and Gli2A groups, the percentage of lung CD4+ cells increased with HDM-treatment (FIG. 5a), indicating T-cell recruitment to the tissue during the response. There was more eosinophil infiltration into the airways in the HDM-treated Gli2A group compared to WT (FIG. 5b), indicating increased severity of the inflammatory phase of disease. In addition, supernatants from BAL were analysed by ELISA for Th2 cytokines IL-4 and IL-13. There were increased levels of both cytokines in the HDM-treated Gli2A group compared to WT (FIG. 5c, d) despite the decreased percentage of CD4+ cells in the lung (FIG. 5a). Blind-scoring of Periodic acid Schiff staining showed that the prevalence of mucus-secreting cells in the bronchioles of Gli2A lungs was also increased compared to WT (FIG. 5e, f).

These data show that Gli-dependent transcription skews T-helper cell differentiation towards the Th2 lineage and exacerbates allergic pathology in vivo. The inventors therefore propose that Hh proteins released from tissue can signal to local T-cells, resulting in enhanced Th2 differentiation/function. To confirm that Hh family members are present in WT adult lung, the inventors assayed whole lung homogenate from WT mice following PBS or HDM treatment. Dhh and Shh mRNA was detectable in lung by RT-PCR (FIG. 5g). The inventors found that expression of Shh transcript (FIG. 5h, qPCR) and protein (FIG. 5i, ELISA) was significantly upregulated in lung homogenates from HDM-treated mice compared to control, whereas expression levels of Dhh and Ihh were similar between groups (data not shown). These data indicate that Hh proteins are expressed in healthy lung, but that only Shh is upregulated under conditions of allergic lung inflammation.

As the proportions of CD4+ cells in the lung of WT and Gli2A mice were not equivalent between groups, the inventors looked at cytokine production on a per cell basis by intracellular staining. The inventors assessed the proportion of CD4+ cells expressing IFNγ, IL-4 and IL-13 from mLN and lung. The inventors found decreased proportions of mLN T-cells positive for IFNγ in HDM-treated Gli2A compared to WT (FIG. 6a, b). Following HDM treatment, the proportions of CD4+ cells that expressed IL13 in lung (FIG. 6c, d) and IL-4 in mLN (FIG. 6a, b) and lung (FIG. 6c, d) were higher in Gli2A compared to WT. Between PBS-treated groups, a significantly increased proportion of Gli2A cells in mLN and lung expressed Th2 cytokines compared to WT, again suggesting that Hh-mediated transcription biases cells towards the Th2 phenotype even without allergen treatment (FIG. 6). Together these data show an increase in measurable parameters of Th2-associated disease in mice where Hh-dependent transcription is active in T-cells.

### Discussion

The inventors have characterised the effect of T-cell-intrinsic Hh signalling on mature T-helper differentiation. Genome-wide expression profiling revealed significant effects of Hh-dependent transcription on gene expression in T-cells. The prediction of a Th2 bias in the DEG profiles, and the observation that Il4 and Gata3 transcription were induced at higher levels in activated Gli2A and lower levels in Gli2R lymphocytes compared to WT, led the inventors to examine the Th differentiation potential of these cells. They found that even in neutral conditions, cells expressing Gli2A produce more IL-4 and upregulate Gata3 more efficiently than WT, whereas repression of physiological Hh-dependent transcription by Gli2R expression had the opposing effect. Loss of environmental Hh in the Dhh KO spleen gave rise to CD4+ cells with decreased Th2 potential when cultured in vitro. In addition, the inventors found transcription of Il4 and Gata3 was modulated by rShh or anti-Hh mAb in WT cells, showing that Hh has a direct functional effect on expression of these Th2-specifying genes. Expression of Il4, but not Gata3 showed significant upregulation during the early stage of activation (<6 h post-stimulation), suggesting that Il4 transcription is key to their Th2 bias. Maximal Il4 expression does however require TCR signalling, as Il4 is weakly expressed by resting/naive T-cells. However, unstimulated Gli2A samples did show higher levels of Il4 transcript than WT (FIG. 1). It therefore seems likely that Hh signalling renders the Il4 locus permissive for transcription, as Gli2A cells require only a TCR signal of short duration to strongly upregulate Il4 expression.

To elucidate the mechanism behind Hh-dependent Th2 skewing, the inventors employed ChIP to show that Gli2A can directly interact with the Il4 gene, at an important enhancer element in intron 2 (Agarwal, S. & Rao, A. Immunity 9, 765-775, 1998). This region has recently been shown to be a DNase-I hypersensitivity site (HS2), critical for full IL-4-dependent Th2 responses (Tanaka, S. et al. Nat Immunol 12, 77-85, 2011), and necessary for chromatin remodelling essential for lineage-specific Il4 expression. The inventors could not detect direct binding of Gli2 to Gata3 using qPCR assays which span 1 kb up- and downstream of the TSS incorporating regions containing possible Gli binding sites. These data thus indicate that the promotion of Th2 differentiation by Gli2 is directly by transcriptional regulation of Il4, rather than via direct interaction with the Gata3 locus. However, the observation that Gata3 expression remains decreased in Gli2R, even on addition of IL-4 and anti-IFNγ, suggests that inhibition of physiological levels of Hh pathway activation in CD4+ cells influences Th differentiation decisions by regulating expression of additional lineage-specifying genes (FIG. 1).

The inventors tested the relevance of their observations in vivo using a model of a Th2-mediated disease, hypothesising that local T-cells undergoing active Hh signalling would exacerbate disease. The transgenic strains are backcrossed to C57BL/6, which elicits less severe pathology in C57BL/6 mice than in BALB/c, but disease was still readily detectable. Presence of the Gli2A transgene in T-cells measurably exacerbated pathology, in keeping with their in vitro data, which showed that Gli2A predisposes T-cells towards Th2 differentiation. The inventors therefore propose that Hh is a novel non-immune-derived modulator of T-cell responses in peripheral tissues, providing an environmental/stromal influence on T-cell differentiation and plasticity. For example, Shh, secreted by lung epithelium during embryonic development is also expressed in adult lung, fibrotic lung tissue and in several lung cancers (Watkins, D. N. et al. Nature 422, 313-317, 2003). The inventors showed that induction of allergic airways disease leads to upregulation of Shh expression in lung tissue (FIG. 5). The inventors therefore propose that in WT allergic lungs, Shh signalling to T-cells would increase Il4 transcription and production, thus enhancing local Th2 responses, signalling to other immune effector cells, and aggravating the disease.

The reason why some tissues are susceptible to Th2-driven pathology, whereas other tissues are prone to inflammatory Th1/Th17 autoimmunity is unclear, and is likely to be the result of tissue-specific factors. There are a few examples of non-immune molecules released from tissue, rather from local immune cells, that influence Th2 function. Gut and skin also express Hh and components of the Hh signalling pathway under conditions of inflammation and repair. Such tissues are frequently targets of Th2-mediated immune responses including allergic disease.

This study investigated the influence of Hh proteins on normal T-cell differentiation. To understand and develop strategies to treat Hh-dependent haematological and lymphoid malignancies, it is clearly important to understand how Hh proteins regulate processes in healthy cells. The inventors are the first to identify Il4 as a direct transcriptional target of Hh signalling in T-cells, and this is of interest not only because of its pivotal role in T-cell biology, but because of the function of IL-4 in signalling to other cell types during immune responses. Importantly, increased IL-4 in tumour microenvironments can inhibit anti-tumour responses or promote tumour growth (Ziegler, A. et al. Blood 113, 3494-3502, 2009; Li, Z. et al. Cancer Res 68, 8687-8694, 2008). Given that many cancers secrete Hh proteins, promotion of Th2/IL-4 by Hh would alter local immune responses, potentially aiding tumour evasion. The observation that Hh signalling in T-cells skewed the local immune response by altering the transcriptional control of Th2 differentiation is therefore an important one.

1. A method of treating, ameliorating or preventing a Th1- or Th2-mediated disease in a subject, the method comprising administering, to a subject in need of such treatment, a therapeutically effective amount of a modulator of Hedgehog (Hh) signaling.

2. The method according to claim 1, wherein the modulator is capable of modulating Sonic Hh (Shh), Indian Hh (Ihh) and/or Desert Hh (Dhh) signalling.

3. The method according to claim 1, wherein the modulator is a negative modulator of Hedgehog (Hh) signalling, which is used to treat a Th2-mediated disease, wherein the modulator comprises an anti-Hh antibody or an Hh inhibitor, which is capable of altering conformation/stability of the receptor through which Hh signalling is directed, or blocking the receptor's activity.

4. (canceled)

5. The method according to claim 3, wherein the Th2-mediated disease is a Th2 inflammatory disease, cancer, chronic lung disease, asthma, scleroderma, allergy, rhinitis, dermatitis, uticaria, anaphylaxis, atrophy or transplant rejection.

6. (canceled)

7. The method according to claim 3, wherein the negative modulator of Hedgehog (Hh) signalling is used to treat asthma.

8. The method according to claim 3, wherein the negative modulator of Hedgehog (Hh) signalling is used to treat Th2-driven cancer.

9. The method according to claim 1, wherein the modulator is a positive modulator of Hedgehog (Hh) signalling, which is used to treat a Th1-mediated disease.

10. The method according to claim 9, wherein the modulator comprises Shh, Ihh or Dhh, or a functional variant or fragment thereof.

11. The method according to claim 9, wherein the modulator comprises a protein comprising an amino acid sequence substantially as set out in SEQ ID No: 2, 4 or 6, or a functional variant or fragment thereof.

12. (canceled)

13. The method according to claim 11, wherein the protein encoded by a nucleic acid sequence substantially as set out in SEQ ID No: 1, 3 or 5, or a functional variant or fragment thereof.

14. The method according to claim 9, wherein the Th1-mediated disease is a Th1 inflammatory disease or is selected from: rheumatoid arthritis (RA); psoriatic arthritis; psoriasis; inflammatory bowel syndrome (IBD); Crohn's disease; ulcerative colitis; multiple sclerosis (MS); flu, including pandemic flu; respiratory disorders for example those caused by viruses such as respiratory syncytial virus (RSV); cystic fibrosis (CF); herpes including genital herpes; sepsis and septic shock; bacterial pneumonia; bacterial meningitis; dengue hemorrhagic fever; diabetes Type I; endometriosis; prostatitis; uveitis; uterine ripening; alopecia areata; ankylosing spondylitis; coeliac disease; dermatomyositis; diabetes mellitus Type 1; Goodpasture's syndrome; Graves' disease; Guillain-Barré syndrome; juvenile idiopathic arthritis; Hashimoto's thyroiditis; idiopathic thrombocytopenic purpura; Lupus erythematosus; mixed connective tissue disease; myasthenia gravis; narcolepsy; osteoarthritis; pemphigus vulgaris; pernicious anaemia; polymyositis; primary biliary cirrhosis; relapsing polychondritis; Sjögren's syndrome; temporal arteritis; vasculitis; Wegener's granulumatosis; and age-related macular degeneration.

15. (canceled)

16. A Th1- or Th2-mediated disease treatment composition comprising a modulator of Hedgehog (Hh) signalling and a pharmaceutically acceptable vehicle, wherein the modulator is as defined in claim 3.

17. (canceled)

18. (canceled)

19. A composition according to claim 16, which is an asthma treatment composition.

20. A composition according to claim 16, which is a Th2-driven cancer treatment composition.

21. An adjuvant comprising a positive modulator of Hedgehog (Hh) signaling, wherein the positive modulator is as defined in claim 10.

22. (canceled)

23. (canceled)

24. A vaccine comprising the adjuvant according to claim 21.

25. (canceled)

26. (canceled)

27. A method of eliciting, in a subject, an effective immune response, the method comprising administering, to a subject, an effective amount of the vaccine according to claim 24.

28. (canceled)

29. A Th1- or Th2-mediated disease treatment composition comprising a modulator of Hedgehog (Hh) signalling and a pharmaceutically acceptable vehicle, wherein the modulator is as defined in claim 11.

30. An adjuvant comprising a positive modulator of Hedgehog (Hh) signaling, wherein the positive modulator is as defined in claim 11.
